(19) United States
(12) Patent Application Publication
(10) Pub. No.: US 2022/0096109 A1
(43) Pub. Date: Mar. 31, 2022

(54) MEDICAL FORCEPS

(71) Applicant: MANI, Inc., Tochigi (JP)

(72) Inventors: Yoshiyuki TAZAWA, Tochigi (JP); Keisuke TSUNODA, Tochigi (JP); Etsuo MURAKAMI, Tochigi (JP); Masamichi SUZUKI, Tochigi (JP)

(21) Appl. No.: 17/392,872

(22) Filed: Aug. 3, 2021

(30) Foreign Application Priority Data
Sep. 28, 2020 (JP) 2020-162055

(51) Int. Cl. A61B 17/28; A61B 90/00

(52) U.S. Cl. CPC A61B 17/282; A61B 2090/0811; A61B 90/08

(57) ABSTRACT

Medical forceps include a pair of arms; and a gripping portion provided at a tip end of each arm, wherein the gripping portion has, at least at part thereof, a first region having a first surface roughness, a region adjacent to the first region has a second region having a surface roughness different from the first surface roughness, and the medical forceps further have, between the first region and the second region, such a light-dark difference that a boundary between the regions is recognizable under illumination.

### CROSS-REFERENCE TO RELATED APPLICATION

This application claims priority from Japanese Patent Application No. 2020-162055 filed with the Japan Patent Office on Sep. 28, 2020, the entire content of which is hereby incorporated by reference.

### BACKGROUND

### 1. Technical Field

One aspect of the present disclosure relates to medical forceps.

### 2. Description of the Related Art

In an ophthalmic surgery, extremely-fine treatment is performed using forceps. Specifically, in a vitreous surgery, treatment is performed for a gripping target object of several μm in some cases. Thus, fine operation of the forceps is required. As these forceps, ophthalmic forceps described in JP-A-2020-044289 have been known. This literature discloses a gripping device in which the visibility of gripping arms is improved.

### SUMMARY

Medical forceps include a pair of arms; and a gripping portion provided at a tip end of each arm, wherein the gripping portion has, at least at part thereof, a first region having a first surface roughness, a region adjacent to the first region has a second region having a surface roughness different from the first surface roughness, and the medical forceps further have, between the first region and the second region, such a light-dark difference that a boundary between the regions is recognizable under illumination.

### BRIEF DESCRIPTION OF THE DRAWINGS

FIG. 1 is an enlarged perspective view of tip ends of medical forceps according to a first embodiment;

FIG. 2 is an enlarged perspective view of tip ends of medical forceps according to a second embodiment;

FIG. 3 is an enlarged perspective view of tip ends of medical forceps according to a third embodiment;

FIG. 4 is an enlarged perspective view of tip ends of medical forceps according to a fourth embodiment; and

FIGS. 5A to 5C are views for describing motion of the forceps according to the first to fourth embodiments, FIG. 5A showing the forceps in a state in which gripping portions are opened, FIG. 5B showing the forceps in a state in which the gripping portions are, on a tip end side thereof, closed slightly, and FIG. 5C showing the forceps in a state in which the gripping portions are closed.

### DESCRIPTION OF THE EMBODIMENTS

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

It is important to enhance the visibility of the tip end portions of forceps so that the accuracy of the fine treatment is further improved. That is, most of the ophthalmic surgeries are performed during observation with a microscope. At this point, an operator can finely perform operation as long as the tip end positions of the forceps is accurately recognizable. For this reason, improvement of the visibility of the tip end portions of the forceps has been demanded.

Specifically, in the fine treatment for the gripping target object of several μm, the accuracy of gripping of the target object is greatly influenced by whether or not the gripping portions at the tip ends of the forceps is accurately recognizable, and such recognition is a factor for determining the accuracy of the treatment.

As the configuration for improving the visibility of the gripping arms, a configuration in which the roughness and reflectance of the gripping arms are changed as disclosed in JP-T-2011-523877 has been known. However, even with the changed roughness of the entirety of the gripping arms, it is difficult to obtain the effect of enhancing the visibility in the treatment requiring accurate gripping in the order of several μm.

One object of the present disclosure is to provide medical forceps useful for improving the visibility of gripping portions at tip ends of forceps.

Medical forceps (present medical forceps) of one aspect of the present disclosure includes a pair of arms; and a gripping portion provided at a tip end of each arm, wherein the gripping portion has, at least at part thereof, a first region having a first surface roughness, a region adjacent to the first region has a second region having a surface roughness different from the first surface roughness, and the medical forceps further have, between the first region and the second region, such a light-dark difference that a boundary between the regions is recognizable under illumination.

As described above, the surface roughness of the gripping portion is changed, and accordingly, a visual difference is caused under illumination. Thus, the accurate shape and position of the gripping portion is recognizable as a light-dark contrast.

The surface roughness described herein means the roughness of a surface of a specific region where illumination light is scattered by fine and irregular protrusions of the surface. The surface roughness is different from a regular recessed-raised shape intended for friction with a gripping target object or an anchor effect. In the case of such a regular recessed-raised shape, it is difficult to obtain a sufficient light-dark contrast under illumination, and it is also difficult to recognize the boundary between the gripping portions under illumination. For this reason, it is difficult to accurately specify the shape and position of the gripping portion.

In the present medical forceps, the first region may be a gripping surface configured to contact and sandwich the treatment target object.

The surface roughness of the gripping surface is changed as described above, so that the positions of the regions contacting and sandwiching the gripping target object is accurately recognizable. Thus, even for fine treatment for a gripping target object of several μm, the present medical forceps can be operated with a high accuracy without degradation of the original functionality of the present medical forceps.

Note that the gripping portion is a region corresponding to the gripping surface. The gripping portion may include, in addition to the gripping surface, a back surface of the gripping surface and the side surfaces between the gripping surface and the back surface. Alternatively, the gripping portion may include only a tip end portion of the gripping surface. As another alternative, the gripping portion may include a peripheral region from which the position of the gripping surface even in the vicinity of a boundary between the gripping surface and the arm portion is substantially recognizable.

In the present medical forceps, the first region may have a rough surface, and the second region may have a mirror surface.

The gripping portion has the rough surface and the adjacent region has the mirror surface as described above, and therefore, the gripping force for the gripping target object can be provided to the gripping portion while the light-dark contrast can be maximized.

From the roughened gripping portion, part of reflected light by scattering reaches the field of view of an operator. Thus, the gripping portion looks shining white. On the other hand, in the mirror-finished adjacent region, light is reflected in a reflection angle direction with respect to an incident angle. Thus, this adjacent region is less likely to be in the field of view of the operator, and looks dark. As a result, the light-dark contrast is caused in, e.g., the field of microscope under illumination. Consequently, the gripping portion becomes noticeable, and the visibility of the gripping portion is improved.

In terms of the gripping portion being noticeable, the mirror-finished region is preferably sufficiently wider than the roughened region. In terms of reduction in friction between the arm portion and an affected area or reduction in friction between the arm portion and a sliding mechanism, the entirety of the arm portion is preferably mirror-finished. Note that roughening and mirror finish can be implemented by well-known roughening and mirror-finish processes.

According to the present disclosure, the medical forceps useful for improving the visibility of the gripping portions at the tip ends of the forceps can be provided.

Hereinafter, embodiments of the present disclosure will be described with reference to the attached drawings. Note that the structure and mechanism of the entirety of forceps are similar to forceps disclosed in JP-A-2020-044289, and therefore, description thereof will be omitted. In description below, description of tip end portions as a main portion of the present embodiments will be focused.

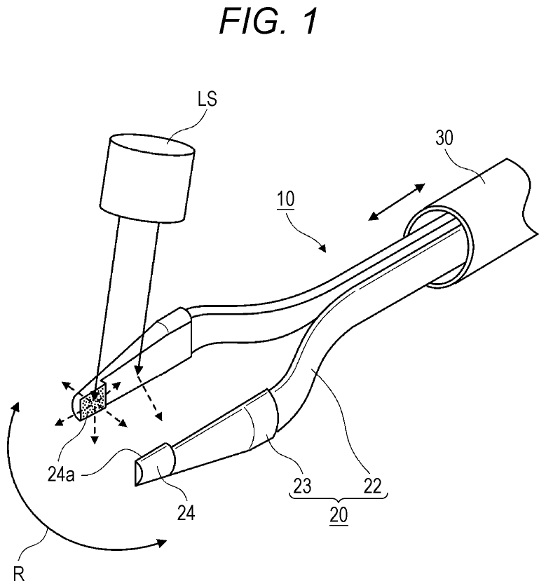

FIG. 1 is an enlarged perspective view of tip ends of medical forceps according to a first embodiment. These medical forceps are used for a medical surgery, for example. As shown in this figure, forceps 10 as the medical forceps according to the first embodiment include neck portions 22 and slit formation portions 23 forming a pair of arm portions 20, gripping portions 24 provided at tip ends of the arm portions 20, and a sleeve 30 arranged on bases of the arm portions 20.

The forceps 10 have a mechanism configured to open/close the pair of gripping portions 24 by slide action when the pair of neck portions 22 moves in or out of an inner cavity of the sleeve 30.

The pair of slit formation portions 23 is provided on a tip end side of the pair of neck portions 22. The pair of gripping portions 24 is provided at ends of the slit formation portions 23. Gripping surfaces 24a are provided on the opposing sides of the gripping portions 24. In association with opening/closing of the gripping portions 24, a gripping target object is brought into contact with the gripping surfaces 24a, and is sandwiched by the gripping surfaces 24a. This gripping surface 24a is one example of a first region provided at least at part of the gripping portion 24 and having a first surface roughness. The gripping surface 24a has a rectangular long flat surface extending in an axial direction. Curved shapes of the neck portions 22 are set such that the gripping surfaces 24a come into surface contact with each other when the gripping portions 24 are closed.

The slit formation portions 23 is provided between the gripping portion 24 and the neck portion 22. These slit formation portions 23 have such shapes that a slit is formed between the pair of slit formation portions 23 when the gripping portions 24 are closed.

With this formed slit, a gripping force on the gripping surface 24a per unit area can be increased. The formed slit also serves as a indication when the gripping portions 24 are visually checked.

The neck portion 22 is formed to have a thickness suitable for elastic deformation. The slit formation portions 23 is formed to have a thickness providing such stiffness that the gripping force is transmittable to the gripping portion 24.

A rough surface region with irregular protrusions is formed at each gripping surface 24a of the gripping portions 24 by roughening, and each gripping surface 24a has the first surface roughness. Part of the gripping portion 24 other than the gripping surface 24a and the entirety of the arm portion 20 are one example of a region adjacent to the gripping surface 24a as the first region, and are also one example of a second region having a surface roughness different from the first surface roughness. A mirror surface region is, by mirror finish, formed at part of the gripping portion 24 other than the gripping surface 24a and the entirety of the arm portion 20.

With these rough and mirror surfaces, light emitted from a light source LS as indicated by solid lines in FIG. 1 causes reflected light indicated by dashed lines in this figure. That is, the reflected light is scattered on the rough surface region. On the other hand, the reflected light is reflected on the mirror surface region in a specific direction. As a result, there is a light-dark contrast between the gripping surface 24a and a portion other than the gripping surface 24a. As described above, the forceps 10 have, between the gripping surface 24a (the first region) and the portion (the second region) other than the gripping surface 24a, such a light-dark difference that a boundary between the regions (e.g., a boundary between the gripping surface 24a and the portion other than the gripping surface 24a) is recognizable under illumination. With this configuration, the visibility of the gripping portion 24 and the gripping surface 24a is improved because the gripping surface 24a is noticeable. Thus, the surface defining region, shape, and size of the gripping surface 24a and the existence position of the gripping surface 24a in a treatment space is accurately recognizable.

Specifically, even in a case where treatment is provided while the arm portions 20 are being rotated in a direction indicated by an arrow R in FIG. 1, i.e., in a clockwise direction or a counterclockwise direction, the gripping surface 24a has the light-dark contrast at an angle other than the rotation angle of the arm portions 20 at which the gripping surface 24a is positioned as a back surface. Thus, the visibility of the gripping portions 24 and the gripping surfaces 24a is improved in many scenes regardless of the rotation posture of the arm portions 20.

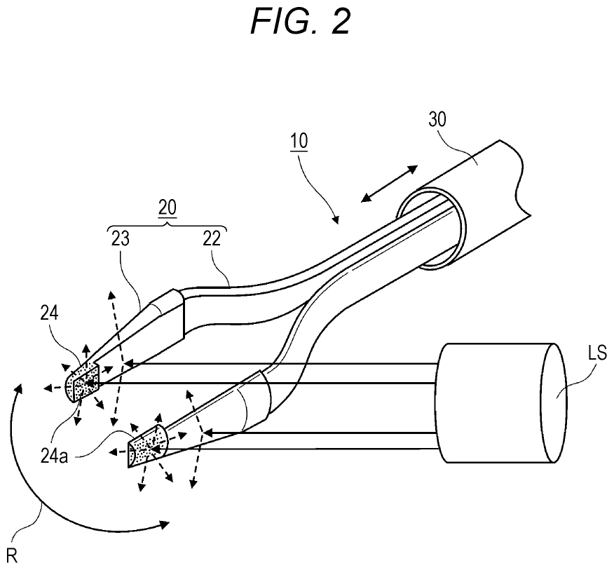

FIG. 2 is an enlarged perspective view of tip ends of medical forceps according to a second embodiment. In the embodiment shown in this figure, a rough surface region (a first region) is also provided at side, end, and back surfaces of a gripping portion 24 in addition to a gripping surface 24a. With this configuration, the gripping portion 24 is recognizable even in a case where the gripping surface 24a is hidden according to the position of a light source LS or a light emission direction and a case where the gripping surface 24a is not exposed by closing of the pair of gripping portions 24.

Note that in a case where the entirety of the gripping portion 24 is roughened, a boundary indicating the surface defining region of the gripping surface 24a is less recognizable. For this reason, for specifically recognizing the gripping surface 24a, only the gripping surface 24a of the gripping portion 24 is preferably roughened as in FIG. 1.

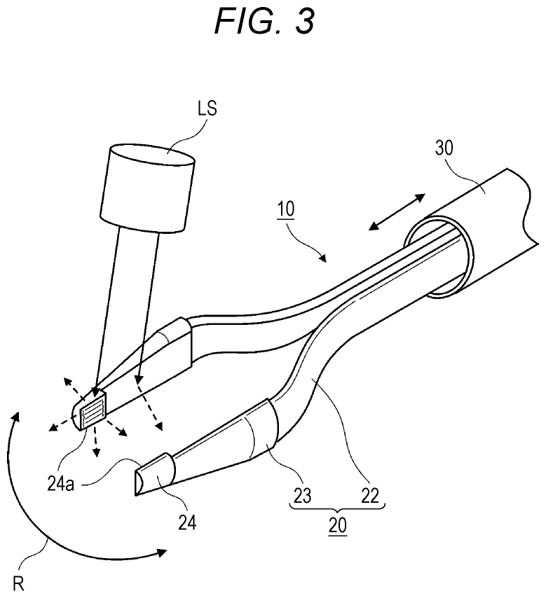

FIG. 3 is an enlarged perspective view of tip ends of medical forceps according to a third embodiment. The embodiment shown in this figure is an example where a rough surface formed at a gripping surface 24a is a rough surface protruding in a direction perpendicular to an axial direction. It is important to form these irregular protrusions in the direction perpendicular to the axial direction for improvement of the visibility of the gripping surfaces 24a of the forceps 10. That is, the protrusions face the direction of light entering from a light source LS of FIG. 3, and therefore, suitable scattering is allowed. Thus, the rough surface formed at the gripping surface 24a is preferably a rough surface including at least the protrusions in the direction perpendicular to the axial direction.

In a case where light enters toward regular protrusions, light is reflected only in a specific direction. For this reason, in a case where the rough surface formed at the gripping surface 24a includes many regular protrusions, the degree of improvement of the visibility of the gripping surfaces 24a depends on the posture of the forceps 10. Thus, the rough surface formed at the gripping surface 24a preferably includes the protrusions with irregular protrusion heights and intervals.

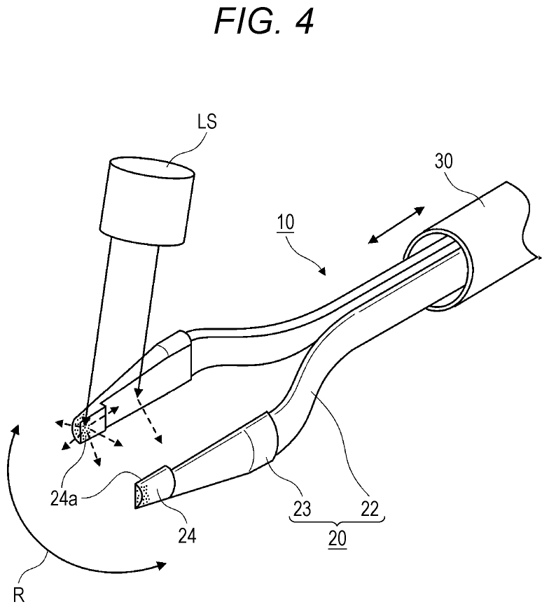

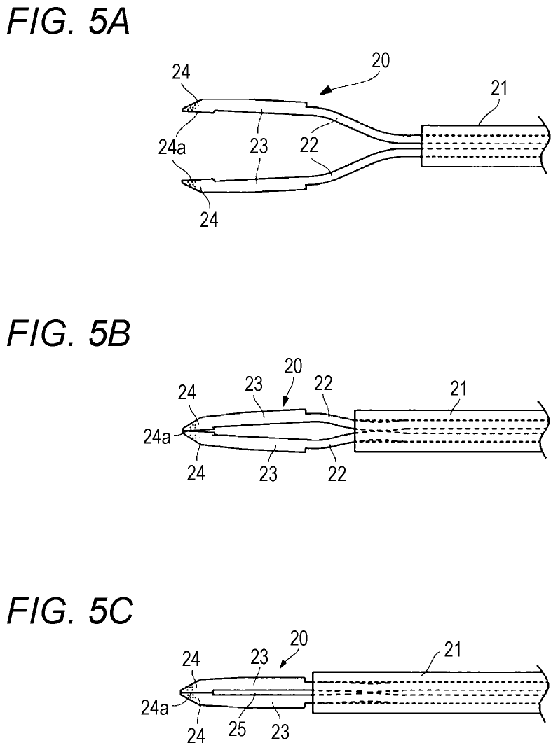

FIG. 4 is an enlarged perspective view of tip ends of medical forceps according to a fourth embodiment. The embodiment shown in this figure is an example where a rough surface region is formed only on a tip end side of a gripping portion 24. This gripping portion 24 is useful for a case where the forceps 10 have, as described later in detail with reference to FIG. 5, such a gripping mechanism that gripping surfaces 24a start contacting a target object from the tip end side when gripping the target object. Moreover, this gripping portion 24 is also useful for specifically recognizing a tip end portion configured to contact the target object first.

FIGS. 5A to 5C are views for describing motion of the medical forceps according to the first to fourth embodiments. FIG. 5A shows the forceps in a state in which the gripping portions are opened. FIG. 5B shows the forceps in a state in which the gripping portions are, on the tip end side thereof, closed slightly. FIG. 5C shows the forceps in a state in which the gripping portions are closed. As in the rough surface regions described in the fourth embodiment, the rough surface regions of these forceps are formed only on the tip end side of the gripping portions 24.

As shown in FIG. 5A, when the neck portions 22 are not housed in the inner cavity of the sleeve 30, the gripping portions 24 are in an open state. When the sleeve 30 is slid from this state to house the leading curved portions of the neck portions 22 in the inner cavity of the sleeve 30, the gripping surfaces 24a of the gripping portions 24 start contacting each other from the tip end side as shown in FIG. 5B. At this point, the tip end side (the tip end side of the gripping portions 24) of the arm portions 20 is in a slightly-closed state. On the other hand, a sleeve side (a sleeve side of the gripping portions 24) of the arm portions 20 is in a slightly-open state.

As shown in FIG. 5C, when the sleeve 30 is further slid to house the entirety of the neck portions 22 in the inner cavity of the sleeve 30, the curved portions on the tip end side function as springs, and accordingly, the gripping surfaces 24a come into surface contact with each other.

According to such a gripping mechanism, in the case of performing fine treatment such as pulling up of a proliferative membrane near a retina, the gripping surfaces 24a can come into surface contact with each other while opening of the gripping surfaces 24a on the tip end side thereof is suppressed. Thus, a microstructure can be suitably gripped by the gripping surfaces 24a.

At a stage before the target object is gripped as shown in FIG. 5A, the tip ends of the gripping surfaces 24a configured to contact the target object first is recognizable by the rough surface regions provided at the tip ends of the gripping surfaces 24a. On the other hand, at a stage at which the target object is gripped as shown in FIGS. 5B and 5C, the tip ends of the gripping surfaces 24a is recognizable by the rough surface regions provided on the back and lateral sides of the gripping surfaces 24a.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. Medical forceps comprising:
a pair of arms; and
a gripping portion provided at a tip end of each arm,
wherein the gripping portion has, at least at part thereof, a first region having a first surface roughness,
a region adjacent to the first region has a second region having a surface roughness different from the first surface roughness, and
the medical forceps further have, between the first region and the second region, such a light-dark difference that a boundary between the regions is recognizable under illumination.

2. The medical forceps according to claim 1, wherein
the first region is a gripping surface configured to contact and sandwich a treatment target object.

3. The medical forceps according to claim 1, wherein
the first region has a rough surface, and the second region has a mirror surface.
